(19) United States
(12) Patent Application Publication
(10) Pub. No.: US 2009/0075325 A1
(43) Pub. Date: Mar. 19, 2009

(54) SYSTEMS AND METHODS FOR ANALYZING AGRICULTURAL PRODUCTS

(75) Inventors: Pradip K. Das, Olivette, MO (US); Joel E. Ream, St. Louis, MO (US); Luis A. Jurado, St. Louis, MO (US)

Correspondence Address: HARNESS, DICKEY & PIERCE, P.L.C., 7700 BONHOMME AVENUE, SUITE 400, ST. LOUIS, MO, 63105, US

(73) Assignee: Monsanto Technology LLC, St. Louis, MO (US)

(21) Appl. No.: 12/234,283

(22) Filed: Sep. 19, 2008

Related U.S. Application Data
(60) Provisional application No. 60/973,692, filed on Sep. 19, 2007.

(51) Int. Cl. G01N 33/50; A01H 5/00; G01N 27/26; G01J 3/30; G01N 1/00; H01J 49/26; G01N 24/08; G02B 21/06; G01N 23/225

(52) U.S. Cl. 435/40.52; 800/298; 204/606; 356/326; 73/23.41; 250/288; 324/318; 359/385; 250/311

(57) ABSTRACT

A method for analyzing agricultural products at a point of transaction is provided. The method comprises presenting a sample comprising at least one seed to a portable analysis system; analyzing the sample for at least one relevant attribute; and characterizing the sample for the transaction based upon the results of the analysis for the at least one relevant attribute.

### CROSS-REFERENCE TO RELATED APPLICATION

This application claims priority from U.S. Provisional Application Ser. No. 60/973,692 (filed Sep. 19, 2007), which is incorporated herein by reference in its entirety.

### FIELD

The present disclosure generally relates to systems and methods for analyzing agricultural products.

### BACKGROUND

The statements in this section merely provide background information related to the present disclosure and may not constitute prior art.

As seed companies continue to introduce a variety of traits into agricultural crops that provide unique compositions and increasing value to the grain and/or its downstream products, there is an increasing need for more sophisticated analysis systems and methods for detecting the traits throughout the value chain (i.e., research and development, seed production, grain production, and grain processing). One such example is oil seeds. Oil seeds are valuable crops with many nutritional and industrial uses due to their unique chemical composition. Accordingly, seed breeders are continually trying to develop varieties of oil seeds to maximize oil seed yield and/or production. As such, grain handlers and seed breeders must be able to distinguish an oil seed from a regular seed to make important decisions in a grain handling situation or in a seed breeding operation. Such decisions have traditionally been based on statistical sampling of a population of seeds because determining the fatty acid characteristics of a population of seeds has been laborious and time consuming. However, statistical sampling necessarily allows some seeds without the desirable trait to remain in the population, and also can inadvertently exclude some seeds from the desired population.

Thus, there is a need for systems and methods providing for the high throughput screening and identification of traits in agricultural products.

### SUMMARY

This section provides a general summary of the disclosure, and is not a comprehensive disclosure of its full scope or all of its features.

This disclosure relates to systems and methods for analyzing an agricultural product at a point of transaction such as a point of delivery or in the marketplace. More specifically, the disclosure provides for the high throughput screening and identification of traits in agricultural products using systems and techniques for portable, high-throughput grain sampling and analysis.

In one embodiment, the disclosure provides for a method of analyzing agricultural products at a point of transaction. The method comprises presenting a sample comprising at least one seed to a portable analysis system, analyzing the sample for at least one relevant attribute; and characterizing the sample for the transaction based upon the results of the analysis for the at least one relevant attribute.

In another embodiment, the disclosure provides for a portable system for analyzing agricultural products. The system comprises a sample presentation module for accepting a sample comprising a plurality of seeds; a grinding module for grinding the sample; at least one instrument for determining at least one relevant attribute of the sample; a communication module for communicating the determined property to a user; and a data management module for analyzing or archiving sample data. In a particular arrangement, an exemplary portable analysis system for identifying premium soybeans can comprise a gas chromatograph in combination with a near-infrared spectrometer.

In still another embodiment, the disclosure provides a method for high throughput screening of oil seeds. The method comprises providing a tissue sample from an oil seed; analyzing the tissue sample with a near-infrared imaging device to obtain a spectral signature of the tissue sample; contacting the tissue sample with a solvent to form a mixture comprising fatty acid methyl esters; analyzing the mixture of fatty acid methyl esters from the sample to determine the fatty acid profile of the corresponding seed; and comparing the spectral signature and the fatty acid profile to spectral signatures and fatty acid profiles of known seeds to determine whether the seed has a desired trait.

Still further, the disclosure provides for a mobile analysis kit useful for identifying premium grain at a point of transaction. The kit comprises a grinding means for preparing a grain tissue sample, a gas chromatograph, a near infrared spectrometer; and a computer comprising software having a calibration model for distinguishing the premium grain from conventional grain.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is an illustration of an exemplary mobile fast gas chromatography instrumentation set-up for use in a system of the present disclosure and including one or more aspects of the present disclosure.

FIG. 2 is an illustration of an exemplary mobile analysis kit including one or more aspects of the present disclosure.

FIG. 3 is a schematic of another exemplary mobile analysis kit including one or more aspects of the present disclosure.

FIG. 4 is a chromatogram of fatty acid esters obtained from a normal soybean in accordance with the method described in Example 1 compared to a chromatogram of fatty acid esters obtained from a low linolenic acid soybean in accordance with the method described in Example 1.

FIG. 5 is a chromatogram of fatty acid esters obtained from a winter oilseed rape seed in accordance with the method described in Example 7.

Corresponding reference numerals indicate corresponding parts throughout the several views of the drawings.

### DETAILED DESCRIPTION

The following description is merely exemplary in nature and is not intended to limit the present disclosure, application, or uses. Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, assemblies, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The present disclosure provides methods for screening agricultural products at a point of transaction to determine whether the product contains a desired trait. As such, embodiments of this disclosure are fully transportable such that testing of most or all of the seeds in a population can be completed in the field. Thus, the rapid assays provided by the present disclosure, which typically require less than about 10 minutes total analysis time, are ideally suited for the identity testing of seeds at grain elevators, processing plants, food formulations laboratories and the like or in seed breeding applications where large numbers of small samples must be analyzed to make immediate planting decisions. Accordingly, the systems and methods of the present disclosure greatly speed up the process of evaluating a population of seeds, for example, in making effective purchasing or handling decisions in the field or in making planting decisions when bulking a given seed population in a breeding program so that time and resources are not wasted in growing plants without desired traits.

In one embodiment, a method for analyzing agricultural products at a point of transaction comprises presenting a sample comprising at least one seed to a portable analysis system, analyzing the sample for at least one relevant attribute; and characterizing the sample for the transaction based upon the results of the analysis for the at least one relevant attribute.

It is contemplated that the methods can be used to determine numerous attributes of the agricultural product including compositional traits, physiological traits, morphological traits, and combinations thereof. For example, suitable compositional traits to be determined using the method of the present disclosure may include protein content, oil content, starch content, amino acid content, fatty acid content, and the like.

Further, the attribute, compositional trait, morphological trait, or the like can be used to determine whether the sample possesses a functional characteristic or meets a user specification such as ethanol yield, feed energy value, nutrition value, oxidative stability, and the like.

It is contemplated that the methods and systems of the present disclosure can be used at various points of transaction including, without limitation, grain storage bins, grain transport vehicles (i.e., trucks, barges or ships), grain elevators, breeding stations, agricultural fields (including, for example, seed production fields or research test plots), processing stations, ports, and retail or consumer outlets.

In a particular embodiment, the methods and systems of the disclosure are used at a grain elevator to determine grain quality, grain composition, whether the grain possesses a functional characteristic, or whether the grain meets a user specification (i.e., whether the grain has a sufficient quantity of a particular composition).

In another embodiment, the methods and systems of the disclosure are used at a breeding station to determine the presence or absence of a desired trait (for example, a genetic marker).

In yet another embodiment, the methods and systems of the disclosure are used in an agricultural field to determine nitrogen content, water content, chlorophyll fluorescence, pathogen infestation, or insect infestation.

In another embodiment, the methods and systems of the disclosure are used at a port to identify toxins, allergens, biological agents, metabolites, bacteria, yeast, molds, and the like.

In another embodiment, the methods and systems of the disclosure are used at a retail or consumer outlet to identify toxins, allergens, biological agents, metabolites, bacteria, yeast, molds, and combinations thereof.

Generally, systems according to the present disclosure for use in analyzing agricultural products comprise a sample presentation module for accepting a sample comprising a plurality of seeds; a grinding module for grinding the sample; at least one analysis instrument for determining at least one relevant attribute of the sample; a communication module for communicating the determined property to a user; and a data management module for analyzing or archiving sample data. Preferably, each of the sample presentation module, grinding module, at least one analysis instrument and the communication module are combined to form a portable analysis system comprising one or more mobile units which can be delivered to the above-described points of transaction to provide on-site and real-time analysis of the agricultural products.

The portable analysis system can include any combination of analysis instruments suitable for use in determining the desired sample properties. Examples of such instruments useful in analyzing agricultural products may include, without limitation, an NWR spectrometer, gas chromatograph, liquid chromatograph, mass spectrometer, nuclear magnetic resonance imager, magnetic resonance imager, terahertz imager, confocal microscope, electron microscope, PCR, and gel electrophoresis.

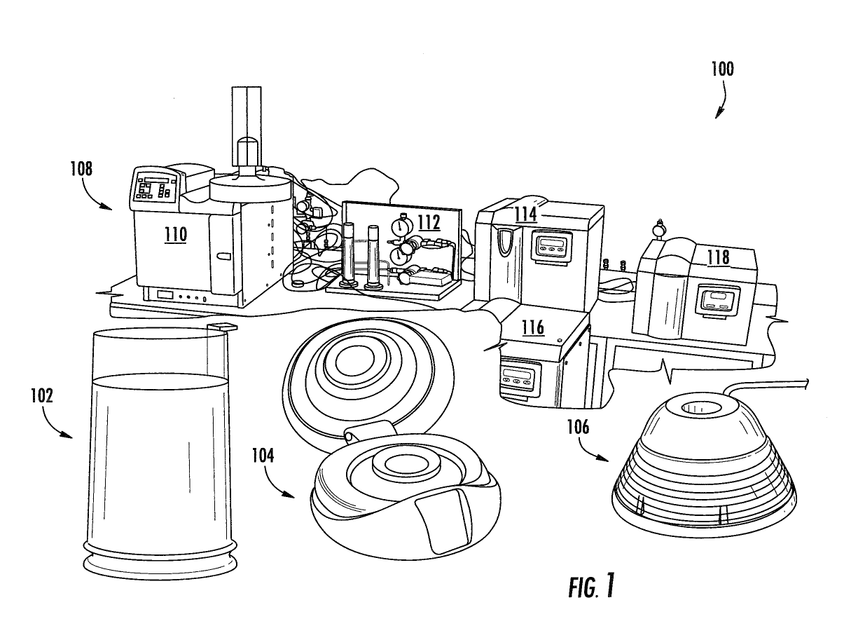

For example, FIG. 1 is an illustration of an exemplary embodiment of a mobile fast gas chromatography instrumentation set-up 100 for use in a system of the present disclosure. The illustrated set-up 100 generally includes a grinder 102, a centrifuge 104, a vortexer 106, and a gas chromatograph assembly 108. The gas chromatograph assembly 108 generally includes a gas chromatograph 110, a gas regulator 112, a hydrogen generator 114, an air compressor 116, and a zero air apparatus 118. The grinder 102, centrifuge 104, and vortexer 106 operate to process a sample and/or prepare a sample for analysis by the gas chromatograph assembly 108. The illustrated set-up 100 is moveable, for example, from a first point of transaction for determining whether a product, sample, etc. at the first point of transaction contains a desired trait to a second point of transaction for determining whether a product at the second point of transaction contains a desired trait.

In another exemplary embodiment, the portable analysis system comprises at least two analysis instruments selected from the group consisting of an NIR spectrometer, gas chromatograph, liquid chromatograph, and a mass spectrometer.

One embodiment of the disclosure for screening oil seeds comprises analyzing the near-infrared spectral signature of the seed along with the fatty acid profile of the seed. For example, such a method generally comprises preparing a spectral signature of the seed using a commercially available near infrared spectrometer from known manufacturers such as Foss, Inc. or Perten Instruments. The method further comprises extracting oil from a seed tissue sample and transesterifying the extracted oils to produce a mixture of fatty acid esters from each sample. The mixture of fatty acid esters is then analyzed by separating and detecting the fatty acid esters to determine a profile of fatty acid characteristics for each sample. The spectral signatures and fatty acid profiles can then be correlated to spectral signatures and fatty acid profiles prepared from seeds of known origin in order to determine the traits of the sampled seed. In an exemplary embodiment, less than about 10 mg of seed tissue, and particularly less than about 5 mg of seed tissue, is sampled from the seed to maintain seed viability as further described below.

The extraction of oils from the sample can be conducted using any suitable solvent known in the art for extracting oil from a seed tissue. Preferably, the selected solvent is suitable for directly extracting and transesterifying oils to a mixture of fatty acid esters. Examples of suitable solvents for the direct extraction and transesterification of oils in the seed sample include without limitation, hexane, benzene, isooctane, tetrahydrofuran, dimethyl sulfoxide, trimethylsulfonium hydroxide, petroleum ether, methylene chloride, and toluene. In an exemplary embodiment, the solvent comprises toluene.

In an exemplary embodiment, the method comprises simultaneously contacting a plurality of seed tissue samples with solvent in individual wells of a multi-well sample plate. For example, to increase throughput and sample handling, samples are preferably contacted with solvent in 96-well or 384-well microtiter plates adapted to accept a volume of solvent sufficient to wet the sample and complete the extraction and transesterification reactions.

The mixture of fatty acid esters produced from the extraction and transesterification reactions is then analyzed to determine the fatty acid characteristics of the individual samples. Such analysis may generally be conducted using any suitable means for separating and detecting the fatty acid esters present in the mixture. Preferably, such separation and detection is completed in less than about 5 minutes, more preferably less than about 3 minutes, so as to maintain throughput. In a particular embodiment, the analysis is conducted using a high speed gas chromatograph with flame ionization detection. An example of such an analysis system is gas chromatography using a Supelco Omegawax column (commercially available from Supelco, Inc., Bellefonte, Pa.). In a further exemplary embodiment, the separation and detection is completed using direct headspace analysis to further increase throughput.

Thus, a particular embodiment for high throughput screening of a seed comprises providing tissue samples from a plurality of seeds in individual compartments of a sample tray; contacting each tissue sample in the sample tray with a solvent to produce a mixture comprising fatty acid esters; and analyzing the mixture of fatty acid esters from each sample to determine the fatty acid profile of the corresponding seeds.

In an exemplary embodiment, the fatty acid profile of the corresponding oil seed is determined in less than about 10 minutes from the time in which an individual tissue sample is contacted with solvent.

The methods and systems of the present disclosure can be used to screen oil seeds such as soybean, corn, canola, rapeseed, sunflower, peanut, safflower, palm and cotton for a wide variety of fatty acid characteristics. For example, in one embodiment, a population of soybeans can be screened to determine the linolenic acid content, stearidonic acid (SDA) content, stearic acid content, oleic acid content, and saturated fat content of individual seeds. In another particular embodiment, a population of rapeseed can be screened to determine erucic acid content, oleic acid content, linolenic acid content, and the saturated fat content of individual seeds. Still further, in another particular embodiment, a population of sunflower can be screened to determine the oleic acid content, stearic acid content, and saturated fat content of individual seeds in the population.

In a particular embodiment, the methods of the present disclosure are used to determine the fatty acid characteristics of seeds in a breeding program. Such methods allow for improved breeding programs wherein nondestructive direct seed sampling can be conducted while maintaining the identity of individuals from the seed sampler to the field. As a result, the breeding program results in a “high-throughput” platform wherein a population of seeds having desired fatty acid characteristics can be more effectively bulked in a shorter period of time, with less field and labor resources required. Such advantages will be more fully described below.

As described above, particular embodiments of the sampling systems and methods of this disclosure protect germination viability of the seeds so as to be non-destructive. Germination viability means that a predominant number of sampled seeds, (i.e, greater than 50% of all sampled seeds) remain viable after sampling. In a particular embodiment, at least about 75% of sampled seeds or at least about 85% of sampled seeds remain viable.

In another embodiment, germination viability is maintained for at least about six months after sampling to ensure that the sampled seed will be viable until it reaches the field for planting. In a particular embodiment, the methods of the present disclosure further comprise treating the sampled seeds to maintain germination viability. Such treatment may generally include any means known in the art for protecting a seed from environmental conditions while in storage or transport. For example, in one embodiment, the sampled seeds may be treated with a polymer and/or a fungicide to protect the sampled seed while in storage or in transport to the field before planting.

The selected seeds may be bulked or kept separate depending on the breeding methodology and target. For example, when a breeder is screening an F₂ population for fatty acid characteristics, all individuals with the desired fatty acid profile may be bulked and planted in the breeding nursery.

Advantages of using the screening methods of this disclosure include, without limitation, reduction of labor and field resources required per population or breeding line, increased capacity to evaluate a larger number of breeding populations per field unit, and increased capacity to screen breeding populations for desired traits prior to planting. Field resources per population are reduced by limiting the field space required to advance the desired phenotypes.

In addition to reducing the number of field rows per population, the screening methods of this disclosure may further increase the number of populations the breeder can evaluate in a given breeding nursery.

The methods of the present disclosure further provide quality assurance (QA) and quality control by assuring that unwanted fatty acid composition characteristics are identified prior to a grain handler making purchasing or processing decisions or a seed breeder making planting decisions.

In an exemplary embodiment, the methods of the present disclosure are used with an automated seed sampler system as described, for example, in U.S. Patent Application Publication No. US2006/0042527, filed Aug. 26, 2005, which is incorporated herein by reference.

In a mobile analytics method of the present invention, the systems and methods may be used to identify premium grain products at various points of transaction during a growing season. For example, using low linolenic soybeans as an illustration, one or more portable analysis units may be transported and deployed at various soybean processing plants at or near anticipated soybean harvest dates. The portable analysis units are then available at the processing plants to identify premium low linolenic soybeans as further described herein. As the local harvest draws to a close, the fully transportable analysis units can then be moved to another soybean processing plant or other point of transaction with a later harvest date. Thus, the portability provided by the systems and method of the present invention allow for real-time analysis at locations when needed without the need to invest significant capital expenses required to build and maintain a permanent laboratory facility.

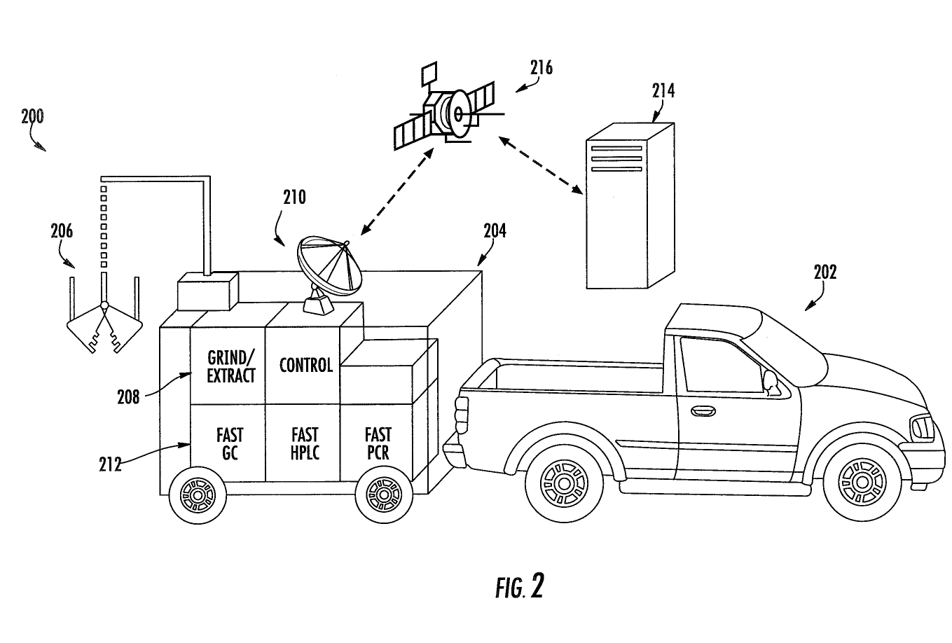

FIG. 2 illustrates an exemplary mobile analysis kit 200 useful, for example, for identifying premium grain, etc. at one or more points of transaction The illustrated kit includes a vehicle 202 and a trailer 204 (together the vehicle 202 and trailer 204 may be considered a mobile platform) coupled to the vehicle for movement. The trailer 204 includes a transfer device 206 for transferring a sample from a storage of sample (e.g., from a soybean truck containing soybeans, etc.) (not shown) to a container device (not shown) on the trailer 204 in preparation for analyzing the sample. The trailer also includes, disposed generally therein, sample preparation equipment 208 (e.g., grinders, extraction equipment, etc.), control equipment 210 (e.g., computer systems, data processing systems, data transfer systems, signal transmitting equipment, etc.), and sample analysis instruments 212 (e.g., GCs, HPLCs, PCRs, NIRs, etc.). The control equipment 210 may operate to communicate with data storage equipment 214 (e.g., via communication links 216, etc.) for storing sample data, further processing sample data, etc. as desired. The illustrated kit 200 is moveable, for example, from a first point of transaction for determining whether a product, sample, etc. at the first point of transaction contains a desired trait to a second point of transaction for determining whether a product at the second point of transaction contains a desired trait. In other exemplary embodiments, kits may include at least one or more additional components and/or at least one or more different components than disclosed herein.

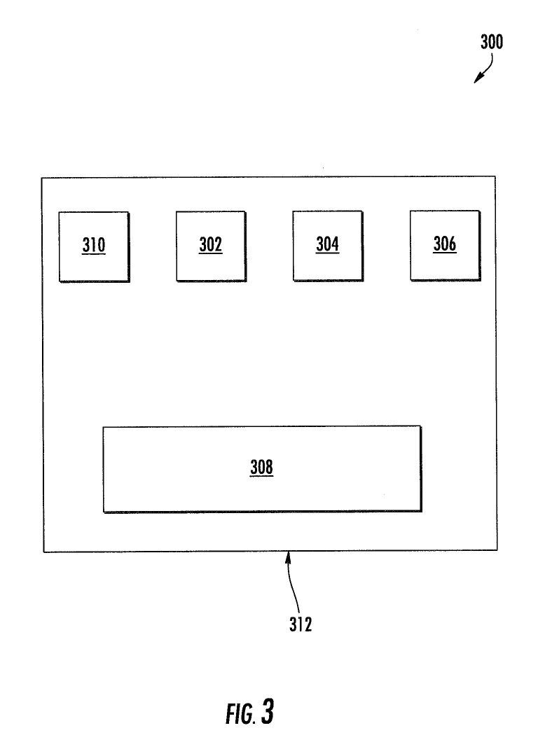

FIG. 3 illustrates another exemplary mobile analysis kit 300 useful, for example, for identifying premium grain, etc. at one or more points of transaction. The illustrated kit 300 generally includes a grinder 302 for preparing a grain tissue sample, a gas chromatograph 304, a near infrared spectrometer 306, and a computer 308 having software with a calibration model for distinguishing, for example, premium grain from conventional grain. The kit 300 may also include one or more extraction solvents 310 for preparing a ground tissue sample for gas chromatography analysis. And the kit 300 may further include a trailer 312 for housing the contents of the kit 300. In other exemplary embodiments, kits may include at least one or more additional components and/or at least one or more different components than disclosed herein.

### EXAMPLES

The following examples are merely illustrative, and not limiting to this disclosure in any way.

### Example 1

This example demonstrates the use of the screening methods of the present disclosure in a program for selection and bulking of Low Linolenic Acid soybeans.

Soybean is the most valuable legume crop, with many nutritional and industrial uses due to its unique chemical composition. Soybean seeds are an important source of vegetable oil, which is used in food products throughout the world. The relatively high level (usually about 8%) of linolenic acid (18:3) in soybean oil reduces its stability and flavor. Hydrogenation of soybean oil is used to lower the level of linolenic acid (18:3) and improve both stability and flavor of soybean oils. However, hydrogenation results in the production of trans fatty acids, which increases the risk for coronary heart disease when consumed. The development of low linolenic acid soybeans has been complicated by the quantitative nature of the trait. The low linolenic acid soybean varieties that have been developed have been found to yield poorly, limiting their usefulness in most commercial settings. Developing a product with commercially significant seed yield is a high priority in most soybean cultivar development programs.

Seed tissue samples (about 5 mg each) were collected from both regular soybean varieties and low linolenic acid soybean varieties and transferred to the individual wells of a 96-well microtiter plate. The samples were then wetted with toluene to extract and transmethylate oil in the samples to produce a mixture of fatty acid methyl esters. The mixture of fatty acid methyl esters were then removed from the wells of the microtiter plate and analyzed on a gas chromatograph.

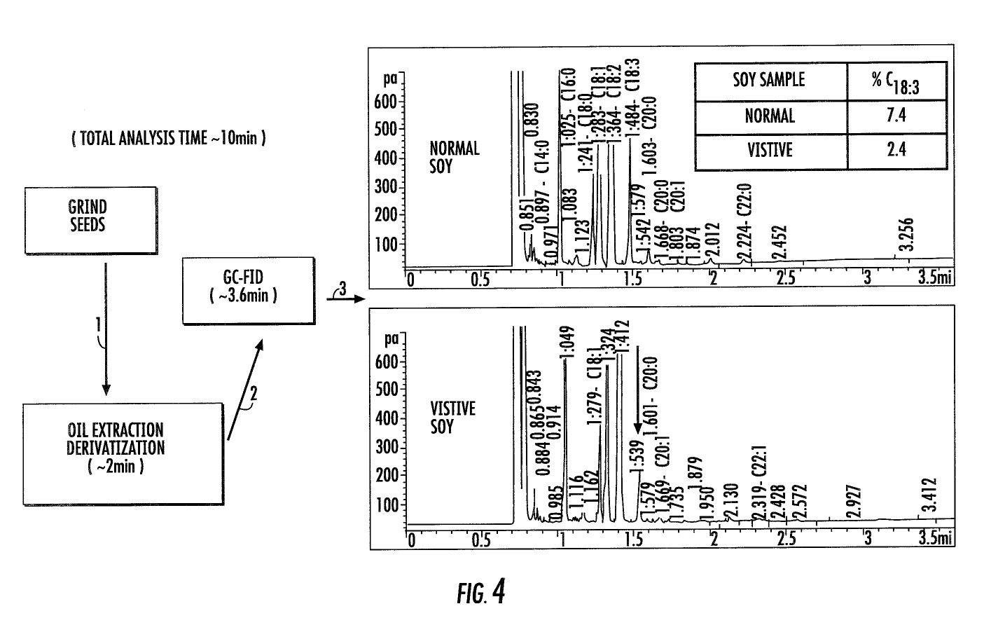

The chromatograph (Supelco Omegawax 320 capillary column using flame ionization detection) was programmed to run in “fast” mode wherein a fast temperature ramp produces a chromatogram in 3.6 minutes. An example of a chromatogram of fatty acid methyl esters for a normal soybean analyzed in the experiment as compared to an example of a chromatogram of fatty acid methyl esters obtained from a low linolenic acid soybean in accordance with this experiment is shown in FIG. 4 (sample preparation and analysis components are also show at reference numbers 1, 2, and 3).

The average fatty acid characteristics for regular soybeans analyzed in this experiment are shown in Table 1.

TABLE 1
Normal Soybeans

| Fatty Acid (% relative) | Average |
|---|---|
| C₁₆ Palmitic acid | 12.8 ± 0.3 |
| C₁₈ Steric acid | 4.2 ± 0.1 |
| C_{18:1n9} Oleic acid | 16.1 ± 1.6 |
| C_{18:2n6} Linolenic acid | 53.5 ± 0.9 |
| C_{18:3} Linolenic acid | 8.8 ± 0.8 |

The average fatty acid characteristics for a low linolenic acid soybeans analyzed in this experiment are shown in Table 2.

TABLE 2
Low Linolenic Soybeans

| Fatty Acid (% relative) | Average |
|---|---|
| C₁₆ Palmitic acid | 10.4 ± 0.3 |
| C₁₈ Steric acid | 4.6 ± 0.4 |
| C_{18:1n9} Oleic acid | 19.3 ± 0.9 |
| C_{18:2n6} Linolenic acid | 59.1 ± 1.0 |
| C_{18:3} Linolenic acid | 3.0 ± 0.3 |

### Example 2

This example demonstrates the use of the screening methods of the present disclosure in a program for selecting Stearidonic Acid (SDA) soybeans.

Tissue samples were collected from soybean varieties identified as 0% SDA, 15% SDA, 20% SDA, and 30% SDA. The tissue samples were contacted with solvent to produce a mixture of fatty acid esters and the fatty acid esters were then separated and analyzed using fast gas chromatography as described in Example 1. The fatty acid profiles of the SDA soybeans are shown in Table 3.

TABLE 3
Fast GC Method and SDA Soybeans

| | | | | 30% |
|---|---|---|---|---|
| Fatty acid (% relative) | 0% SDA | 15% SDA | 20% SDA | SDA |
| C₁₄ Myristic acid | 0 | 0.3 | 0.3 | 0.3 |
| C₁₆ Palmitic acid | 11.9 | 12.5 | 12.7 | 13.1 |
| C₁₈ Steric acid | 3.8 | 3.7 | 3.7 | 3.7 |
| C_{18:1n9} Oleic acid | 20.3 | 15 | 17.1 | 15.3 |
| C_{18:2n6} Linoleic acid | 50.8 | 32 | 28.2 | 17 |
| C_{18:3n6} gamma Linolenic | — | 3.8 | 4.8 | 4.6 |
| C_{18:3} Linolenic acid | 7.7 | 11.1 | 10.5 | 12.2 |
| C_{18:4n3} Octadecatetraenoic | — | 13 | 16 | 26.8 |
| C₂₀ Arachidonic acid | 0.6 | 0.8 | 0.6 | 0.7 |
| C_{20:1n9} Eicosenoic acid | 0.2 | 0.4 | 0.3 | 0.4 |
| C₂₂ Behenic acid | 0.3 | 0.3 | 0.3 | 0.4 |
| C₂₄ Lignoceric acid | 0 | 0.1 | 0.1 | 0.1 |

### Example 3

This example demonstrates the use of the screening methods of the present disclosure in a program for selecting High Stearic Acid soybeans.

Tissue samples were collected from soybean varieties identified as high stearic acid soybeans. The tissue samples were contacted with solvent to produce a mixture of fatty acid esters and the fatty acid esters were then separated and analyzed using fast gas chromatography as described in Example 1. The fatty acid profiles of the high stearic acid soybeans are shown in Table 4.

TABLE 4
High Stearic Acid Soybeans

| Fatty acid (% relative) | Fast GC method |
|---|---|
| C₁₄ Myristic acid | 0 |
| C₁₆ Palmitic acid | 8.9 |
| C₁₈ Steric acid | 20.3 |
| C_{18:1n9} Oleic acid | 21.4 |
| C_{18:2n6} Linoleic acid | 37.8 |
| C_{18:3} Linolenic acid | 3.1 |
| C₂₀ Arachidonic acid | 1.8 |
| C_{20:1n9} Eicosenoic acid | 0.1 |
| C₂₂ Behenic acid | 1.0 |
| C₂₄ Lignoceric acid | 0.2 |

### Example 4

This example demonstrates the use of the screening methods of the present disclosure in a program for screening rapeseed.

Tissue samples collected from rapeseed were contacted with toluene to produce a mixture of fatty acid esters. The fatty acid esters were then separated and analyzed using fast gas chromatography as described in Example 1 The samples were screened and identified as follows: (1) conventional rapeseed (i.e., having an erucic acid content less than about 2%); (2) rapeseed having an erucic acid content greater than about 2%; (3) rapeseed having an erucic acid content of greater than about 45%; (4) rapeseed having an erucic acid content of greater than 45% and a linolenic acid content of less than about 3.5%; (5) rapeseed having a linolenic acid content of less than about 3.5%; (6) rapeseed having an oleic acid content of greater than about 70%; (7) rapeseed having less than about 7% saturated fat; (8) rapeseed having less than about 6% saturated fat; (9) rapeseed having less than about 5% saturated fat; (10) rapeseed having an oleic acid content of greater than about 70% and a linolenic acid content of less than about 3.5%; and (11) rapeseed having an oleic acid content of greater than about 70%, a linolenic acid content of less than about 3.5%, and less than about 7% saturated fat.

### Example 5

This example demonstrates the use of the screening methods of the present disclosure in a program for screening sunflower.

Tissue samples collected from sunflower seeds were contacted with toluene to produce a mixture of fatty acid esters. The fatty acid esters were then separated and analyzed using fast gas chromatography as described in Example 1. The samples were screened and identified as follows: (1) an oleic acid content of from about 40% to about 70%, (2) an oleic acid content of greater than about 70%, (3) a stearic acid content of greater than about 6%, (4) a saturated fat content of less than about 8%, (5) an oleic acid content of greater than about 70% and a saturated fat content of less than about 8%, and (6) an oleic acid content of greater than about 70%, a stearic acid content of greater than about 6%, and a saturated fat content of less than about 8%.

### Example 6

This example demonstrates a system for detecting low linolenic soybeans in a mobile laboratory unit on-site at a grain elevator. The development of low linolenic soybeans for use in preparing trans-fat free vegetable oil has required grain elevators to distinguish low-linolenic soybeans from commodity soybeans. Using the mobile laboratory system of the present disclosure to distinguish low linolenic soybeans allows farmers to receive a premium for growing the low-linolenic soybeans. Low-linolenic soybeans provide soybean processors an opportunity to supply a modified soybean oil to help meet the demand for trans-fat free vegetable oil among food companies and restaurants. The systems and methods described herein assure that soybean processors receive only soybean grain meeting the linolemic acid level specifications that, when processed, yield a trans fat free vegetable oil.

The example comprised using a mobile analysis unit at an Ag Processing Inc. (AGP) soybean processing plant in Mason City, Iowa to identify low linolenic soybeans (soybeans having less than about 3% linolenic acid). The mobile analysis unit comprised a trailer housing seed grinding equipment, a gas chromatograph as described in Example 1 above, a near infrared transmittance (NIT) instrument such as an INFRATEC 1241 grain analyzer commercially available from FOSS North America, Minneapolis, Minn. and software comprising a calibration model to correlate the analysis results in distinguishing between low linolenic and conventional soybeans. Results from the analyses showed good correlation between data from the gas chromatograph and the NIT instrument suggesting that the mobile analysis unit were successful in providing accurate, cost effective analyses to identify premium, low linolenic soybeans in real time at the point of sale and before processing.

### Example 7

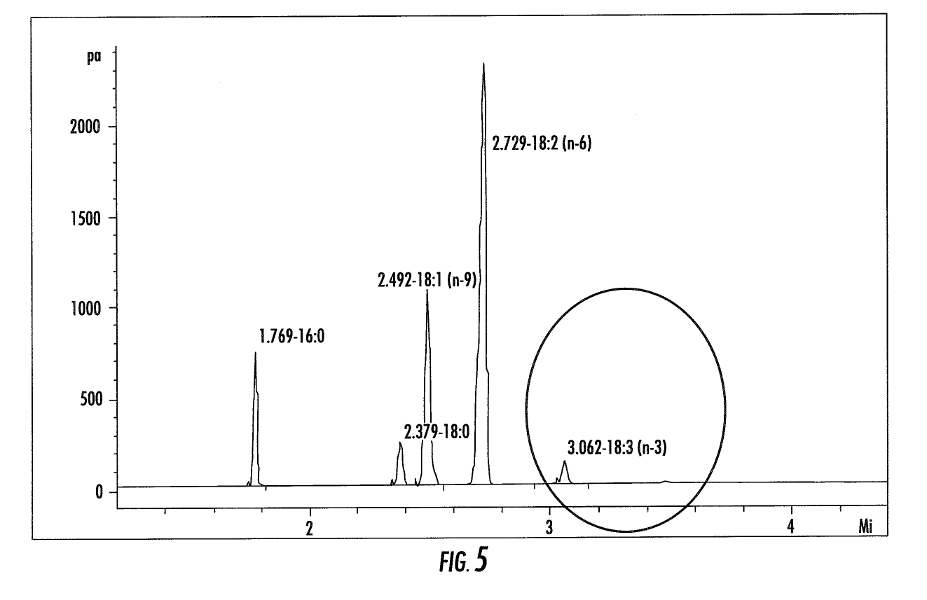

This example demonstrates the use of a fast gas chromatograph system at a grain elevator for identifying Winter Oilseed Rapeseed having low linolenic acid and high oleic acid contents. The experimental procedure, which was accomplished in less than 10 minutes per sample, comprised placing about 100 mg of ground seed tissue in an eppendorf tube. 1.0 mL of isooctane was added to the tube and the sample was mixed by vortexing for 20-30 seconds. The sample was then centrifuged for 1 minute at 3000 rpm. The supernatant comprising extracted oils was transferred to a 1.8 mL glass vial and 0.5 mL of derivatizing agent (Meth Prep II) was added. The sample was again vortexed for 10 seconds wherein the isooctane and methanol separated into two layers. 1 uL from the top layer was injected into a GC programmed for a run time of 3.6 min as described in Example 1. Results are shown in FIG. 5.

When introducing elements of the present disclosure, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, systems, and methods without departing from the scope of the present disclosure, it is intended that all matter contained in the above description and shown in the accompanying drawings shall be interpreted as illustrative and not in a limiting sense. It is also understood that the present disclosure is not limited to the embodiments described above, but encompasses any and all embodiments within the scope of the following claims.

What is claimed is:

1. A method for analyzing agricultural products at a point of transaction, the method comprising:
presenting a sample comprising at least one seed to a portable analysis system;
analyzing the sample for at least one relevant attribute; and
characterizing the sample for the transaction based upon the results of the analysis for the at least one relevant attribute.

2. The method of claim 1, wherein the method further comprises preparing the sample for analysis.

3. The method of claim 2, wherein the step of preparing the sample for analysis comprises grinding the sample.

4. The method of claim 1, wherein the at least one relevant attribute is selected from the group consisting of a compositional trait, a physiological trait, a morphological trait, and combinations thereof.

5. The method of claim 1, wherein the at least one relevant attribute is the presence or absence of a compositional trait.

6. The method of claim 5, wherein the compositional trait is selected from the group consisting of protein content, oil content, starch content, amino acid content, and fatty acid content.

7. The method of claim 4, wherein the method further comprises correlating the at least one relevant attribute to determine whether the sample possesses a functional characteristic or meets a user specification.

8. The method of claim 7, wherein the functional characteristic or user specification is selected from the group consisting of ethanol yield, feed energy value, nutrition value, and oxidative stability.

9. The method of claim 1, wherein the point of transaction is selected from the group consisting of a grain elevator, a seed production field, a breeding station, an agricultural field, a processing station, a port, and a retail or consumer outlet.

10. The method of claim 1, wherein the point of transaction is a grain elevator and the relevant attribute is selected from the group consisting of grain quality, grain composition, whether the grain possesses a functional characteristic, whether the grain meets a user specification, and combinations thereof.

11. The method of claim 1, wherein the point of transaction is a breeding station and the relevant attribute is the presence or absence of a genetic marker.

12. The method of claim 1, wherein the point of transaction is an agricultural field and the relevant attribute is selected from the group consisting of nitrogen content, water content, chlorophyll fluorescence, pathogen infestation, and insect infestation.

13. The method of claim 1, wherein the point of transaction is a port and the relevant attribute is selected from the group consisting of toxins, allergens, biological agents, metabolites, bacteria, yeast, molds, and combinations thereof.

14. The method of claim 1, wherein the point of transaction is a retail or consumer outlet and the relevant attribute is selected from the group consisting of toxins, allergens, biological agents, metabolites, bacteria, yeast, molds, and combinations thereof.

15. A portable system for analyzing agricultural products, the system comprising:
a sample presentation module for accepting a sample comprising a plurality of seeds;
a grinding module for grinding the sample;
at least one instrument for determining at least one relevant attribute of the sample;
a communication module for communicating the determined property to a user; and
a data management module for analyzing or archiving sample data.

16. The system of claim 15 wherein the instrument for determining the sample property is selected from the group consisting of an NIR spectrometer, gas chromatograph, liquid chromatograph, mass spectrometer, nuclear magnetic resonance imager, magnetic resonance imager, terahertz imager, confocal microscope, electron microscope, and gel electrophoresis.

17. The system of claim 15 further comprising at least two instruments selected from the group consisting of an NIR spectrometer, gas chromatograph, liquid chromatograph, and a mass spectrometer.

18. The system of claim 15 wherein the seed is a soybean and the system comprises a gas chromatograph and an NIR spectrometer.

19. The system of claim 15, wherein the system is moveable from at least one point of transaction for determining whether a product at said at least one point of transaction contains a desired trait to at least another point of transaction for determining whether a product at said at least another point of transaction contains a desired trait.

20. A method for high throughput screening of oil seeds, the method comprising:
providing a tissue sample from an oil seed;
analyzing the tissue sample with a near-infrared imaging device to obtain a spectral signature of the tissue sample;
contacting the tissue sample with a solvent to form a mixture comprising fatty acid methyl esters;
analyzing the mixture of fatty acid methyl esters from the sample to determine the fatty acid profile of the corresponding seed; and
comparing the spectral signature and the fatty acid profile to spectral signatures and fatty acid profiles of known seeds to determine whether the seed has a desired trait.

21. The method of claim 20, wherein the step of analyzing the mixture of fatty acid methyl esters comprises separating and detecting the fatty acid methyl esters using gas chromatography.

22. The method of claim 20 wherein the fatty acid profile of the corresponding oil seed is determined in less than about 10 minutes from the time in which the tissue sample is contacted with solvent.

23. The method of claim 20, wherein the oil seeds are selected from the group consisting of soybean, corn, canola, rapeseed, sunflower, peanut, safflower, palm and cotton.

24. The method of claim 20, wherein the seed is soybean and the desired trait is a linolenic acid content of less than about 8%.

25. A mobile analysis kit useful for identifying premium grain at a point of transaction, the kit comprising:
a grinder for preparing a grain tissue sample;
a gas chromatograph;
a near infrared spectrometer; and
a computer comprising software having a calibration model for distinguishing the premium grain from conventional grain.

26. The mobile analysis kit of claim 25 further comprising one or more extraction solvents for preparing a ground tissue sample for gas chromatography analysis.

27. The mobile analysis kit of claim 25 further comprising a trailer for housing the contents of the kit.

28. The mobile analysis kit of claim 25, wherein the system is moveable from at least one point of transaction for determining whether a product at said at least one point of transaction contains a desired trait to at least another point of transaction for determining whether a product at said at least another point of transaction contains a desired trait.
